(19) United States
(12) Patent Application Publication
(10) Pub. No.: US 2001/0020007 A1
(43) Pub. Date: Sep. 6, 2001

(54) Vitamin preparations for reducing oxygen consumption during physical efforts

(76) Inventor: Oswald Wiss, Basel (CH)

Correspondence Address: LEYDIG VOIT & MAYER, LTD, 700 THIRTEENTH ST. NW, SUITE 300, WASHINGTON, DC, 20005-3960, US

(21) Appl. No.: 09/824,801

(22) Filed: Apr. 4, 2001

Related U.S. Application Data
(63) Continuation-in-part of application No. 09/242,614, filed on Feb. 19, 1999.
Application No. 09/242,614 is a 371 of international application No. PCT/CH97/00298, filed on Aug. 14, 1997.

(30) Foreign Application Priority Data
Aug. 26, 1996 (CH) 2097/96

(51) Int. Cl. A61K 38/05; A61K 31/7004

(52) U.S. Cl. 514/19; 514/23; 514/62

(57) ABSTRACT

The invention relates to a method of decreasing oxygen consumption during physical work and to preparations having such effect. The effect is achieved by administering efficient quantities of certain combinations of (a) D-glucose, D-maltose, ethanol, glucogenic amine, glucogenic amino acid or an amino acid metabolizable through glyoxal, or a dipeptide or pharmaceutically acceptable salt of such an amino acid, and (b) a vitamin components selected from thiamine, thiamine salts or combinations of folic acid and cyanocobalamin. Simultaneous administration of the combinations is efficient when taken prior to or during physical efforts or even one day before. Preferably, the combinations are administered in the form of gelled preparations containing a gelling agent.

### RELATED APPLICATION

This application is a continuation-in-part of application Ser. No. 09/242,614, filed Feb. 19, 1999, which is a 371 of PCT/CH97/00298, filed Aug. 14, 1997.

### FIELD OF THE INVENTION

The present invention relates to a method of decreasing oxygen consumption in animal including human during physical work comprising administering certain energy-producing substances together with certain vitamins, and to novel preparations having this action.

### BACKGROUND OF THE INVENTION

It is known that the muscle glycogen is dependent on nutrition and that only certain nutritive constituents are suitable for its synthesis and are active to a differing extent. As a prerequisite for their activity, glucose or glucogenic metabolites must be formed in the course of their reaction in the metabolism. De facto, the activity is restricted to carbohydrates and proteins, as a further prerequisite it being necessary that they are rapidly broken down into their constituents in the alimentary tract and in the course of this glucose and glucogenic amino acids are released which can positively affect muscular glycogen synthesis. If it is broken down metabolically as a result of physical activity, the muscle glycogen affects the oxygen requirement thus caused.

In addition to the glycogen, fats are available to the muscle, which can be utilized on their own or together with the glycogen. In the end, the effect of the two is that ATP (adenosine triphosphate) is cleaved and thus energy is released for muscle contraction. Glycogen and fats differ, however, in that the glycogen, unlike the fats, needs no oxygen for energy production, because the formation of glucose from glycogen is subject to an anaerobic metabolic sequence, while the fatty acids released from fats are utilized aerobically. The oxygen requirement is thus reduced by the breakdown of glycogen, which is stored in the stressed musculature, because its utilization—unlike the utilization of the fatty acids—does not need any molecular oxygen for energy supply.

The efficiency during physical activity is limited by the individual oxygen absorption capacity. Active compounds which, under identical mechanical stress, reduce the oxygen requirement, thus increase the efficiency. The breakdown of the glycogen stored in the muscle in this sense naturally makes possible an increase in the efficiency. After breakdown has taken place, the glycogen is stored again in the resting phase, with the condition that suitable nutrients, such as easily digestible carbohydrates or meat of young animals and fish, are consumed. For example, an evening meal which is rich in easily digestible polysaccharides (e.g. pasta), causes an oxygen consumption which is by up to approximately 30% lower for the same ergometrically measured work during approximately 1 hour on the following day before breakfast than a protein-rich evening meal.

Nutrients which are administered briefly before or during physical work, however, are ineffective or have an adverse effect with respect to the oxygen consumption insofar as they increase the oxygen requirement for the same work. These interactions are to be attributed to additional oxygen requirements, caused by digestion, absorption and anabolism. Glucose forms an exception when it is administered in doses of 5 to 25 g briefly before or during physical stress, which is to be attributed to the fact that the absorption capacity of the working muscle for glucose is up to 50 times higher than in the resting state. The reduction in the oxygen consumption achieved thereby is nevertheless only approximately 1 to 3%.

It is likewise known that certain vitamins are necessary as catalysts for the energy utilization of glucose and fatty acids. Lack of such vitamins results in a decrease in the physical efficiency. For optimum efficiency, regular supply with the recommended daily amounts laid down is sufficient, which depending on the vitamin are in the region of a few milligrams or micrograms. However, it has been shown again and again that the supply of relatively high doses of vitamins, e.g. of vitamin B₁ (thiamine), which has a key function in energy metabolism, does not cause any increase in the normal physical efficiency.

A number of products are already known which, on the one hand, should contain energy-producing components and, on the other hand, the vitamins needed for their metabolism and which should guarantee an optimum energy supply, maintain the anabolism or combat vitamin deficiencies. Furthermore, various pharmaceutical preparations are also known which, in addition to pharmaceutical active compounds, also can contain vitamins and carbohydrates, proteins or ethanol.

For example, U.S. Pat. No. 5,292,538 describes a fructose/glucose/protein mixture which, in addition to glucose polymers, fructose, partially hydrolysed proteins and, if appropriate, a lipid source, contains a magnesium complex and vitamins, such as vitamin A, B₁, B₂, B₅, B₆, B₁₂, C, D, E, folic acid, niacinamide and biotin, and should improve the endurance abilities and the anabolism.

WO-A-90/02489 discloses an energy-producing dietetic product, which by means of combination of fast and slow sugars should make possible an immediate and continuous energy contribution and which is provided with a coating of chocolate containing vitamin B₁ and/or B₂.

FR-A-2,704,392 discloses an absorbable nutritional supplement for improving the physical and mental efficiency, which contains magnesium and the vitamins C and B₁ together with a carrier, which can preferably consist of cellulose, dextrose, magnesium stearate and carrot powder. In this context, the fact should be utilized that vitamin C increases the efficiency of the muscles and, on the other hand, magnesium and vitamin B₁ are needed for the enzymatic breakdown of sugars and fats.

EP-A-0,087,068 discloses a nutritional additive which contains selenium, cysteine, L-tryptophan, L-tyrosine and, if desired, further constituents, such as fructose, vitamin B₁, vitamin C and calcium salts, and which should be suitable for replacing the essential nutritional constituents which have been exhausted as a result of excessive alcohol consumption.

U.S. Pat. No. 5,039,668 describes a composition for the treatment of vitamin deficiencies and as a cough medicine which, in addition to liquid bees honey, histidine, lysine, tryptophan and calcium or iron salts, contains vitamins, such as, for example, vitamin B₁₂ and folic acid or the vitamins B₁, B₆ and B₁₂ as well as niacin.

EP-A-0,482,715 proposes a composition based on protein-free carbohydrates and vegetable fats together with essential amino acids, which should allow a balanced nutritional supply and have an immunostimulating action and which furthermore—on account of specific proportions of the essential amino acids—should make possible a higher NNU value (net nitrogen utilization). The proposed composition—in addition to the amino acids isoleucine, leucine, lysine, methionine, phenylalanine, threonine, tryptophan and valine—contains a carbohydrate from the group consisting of sucrose, maltose and sorbitol, a highly unsaturated vegetable fat from the group consisting of safflower oil, sunflower oil and maize oil, and the vitamins A, B₁, B₂, B₆, B₁₂, C, D, K, biotin, folic acid, α-tocopherol, nicotinamide and pantothenic acid.

In JP-A-07/330583, a liquid preparation is disclosed which is suitable as an enteral nutrient for patients after surgical intervention or burns and which, in addition to amino acids, can preferably also contain mineral salts, dextrin, soya bean oil and vitamin A, B₁, B₂, B₆, B₁₂, C, D, E, K, folic acid and biotin.

JP-A-05/124974 proposes a preparation based on Fomes japonicus, which should promote the breakdown of the glycogen stored in the liver. It can be prepared, for example, in the form of a beverage which, in addition to the fungus extract, can additionally contain maltose, oligosaccharides, folic acid, vitamin C, vitamin B₁₂ and iron.

In JP-A-02/078624, the use of an extract of the fibres of bamboo shoots for the treatment of rheumatism is disclosed and a preparation is described which, in addition to the extract, also contains ethanol, vitamin B₁ and vitamin L.

JP-A-02/078625 describes the use of an extract of Adenophora triphylla together with vitamin B₁ for the treatment of pollinosis and discloses a preparation in ethanol.

JP-A-52/143255 proposes a medicinal beverage for masking the flavour of pharmaceuticals which, in addition to a pharmaceutical, such as garlic, ginseng, cranesbill, vitamin A, B₁, B₂ and the like, contains a beer obtained by surface fermentation and has an alcohol content of 0.2 to 3% by weight.

The vitamin content of the previously known compositions aims either at an adequate or increased supply of certain vitamins or an optimum utilization of nutritional constituents. An oxygen-sparing action is not aimed at, nor suspected. In fact, the known compositions would as a rule also be unsuitable to achieve a significant oxygen-sparing action during physical work, since—as mentioned at the outset—most nutritional constituents are either inactive or—like sucrose, fructose and proteins—even increase the oxygen requirement if they are consumed immediately before or during physical activity.

### SUMMARY OF THE INVENTION

Surprisingly, it has now been found that certain vitamins in combination with certain compounds result in a marked decrease in the oxygen consumption during physical work and thus make possible an increase in the physical efficiency. The oxygen-sparing or performance-increasing action can be determined directly by measurement of the oxygen consumption or of the physical work and is likewise detectable by a reduction in the heart rate with the same work or a higher efficiency at constant heart rate.

The invention therefore relates to the use of (a) an efficacious amount of D-glucose, D-maltose, ethanol, of a glucogenic amine, of a glucogenic amino acid or one which can be metabolized via glyoxylate or of a dipeptide or pharmaceutically acceptable salt of such an amino acid as a first component and of (b) an efficacious amount of thiamine, of a pharmaceutically acceptable thiamine salt or of a combination of folic acid and cyanocobalamin as a second component for the production of a preparation for decreasing the oxygen consumption during physical work, with the proviso that the second component is thiamine or a pharmaceutically acceptable thiamine salt if the first component used is D-glucose, D-maltose, a glucogenic amine, a glucogenic amino acid which cannot be metabolized via glyoxylate, or a dipeptide or pharmaceutically acceptable salt of such an amino acid. The invention further relates to novel preparations having said oxygen-sparing action, and to a method of decreasing oxygen consumption in animal including human during physical work which comprises administering simultaneously to said animal including human efficacious amounts of a first component (a) and a second component (b), as defined above. Said simultaneous administration may be effected by separate but simultaneous administration of components (a) and (b) or by administering a preparation comprising components (a) and (b). All statements made hereinbelow in respect of preparations comprising components (a) and (b) equally apply to separate but simultaneous administration of its components, and vice versa, unless expressly indicated otherwise.

The expression “dipeptide” in the context of the present invention includes the dipeptides of glucogenic amino acids or amino acids which can be metabolized via glyoxylate, in particular dipeptides from two identical amino acids such as H-Gly-Gly-OH, H-Ser-Ser-OH and H-Glu-Glu-OH. The expression “amino acid which can be metabolized via glyoxylate” in the context of the present invention includes, in particular, the amino acids suitable for nucleic acid synthesis.

In a preferred embodiment of the invention the first and the second components are not comprised in a preparation containing magnesium, thiamine, ascorbic acid, cellulose, glucose, magnesium stearate and carrot powder.

Though thiamine or a pharmaceutically acceptable salt thereof, if utilized, can be administered in amounts of up to 1000 mg or more per dose, preferred doses are about 500 mg or less, more preferably about 100 mg or less.

Moreover, the present invention does not require selenium. Therefore, the administrable compositions or combinations of the first and second and optional further components preferably comprise less than 50 μg of selenium per dose; more preferably, they can be essentially free of selenium and, for example, contain less than 1 μg of selenium per dose.

### DETAILED DESCRIPTION

The invention is based on the finding that efficacious doses of thiamine (vitamin B₁), of a pharmaceutically acceptable thiamine salt, such as thiamine mononitrate or thiamine hydrochloride, or of a combination of folic acid (vitamin M) and cyanocobalamin (vitamin B₁₂), if they are taken at the same time with the substances mentioned under (a) at a suitable dose, can replace the nutritional carbohydrates and nutritional proteins, which are needed in amounts of 50 to 200 g per meal, in the function of the stimulation of muscular glycogen formation, and that in this combination doses of, for example, a few grams of component (a) or, in the case of additional use of a gelling agent, even considerably lower doses are sufficient to achieve the desired action. On the other hand, the vitamins on their own are inactive and also the substances mentioned under (a) in the amounts utilizable per se on their own according to the invention do not produce any or produce only a very slight oxygen-sparing or performance-increasing action.

The utilization of the muscular glycogen reserves relieves the load on the heart, lungs and circulation, because less oxygen has to be absorbed for the same physical work and as a result the respiratory and heart rate is reduced. However, limits are placed on the glycogen content of the muscle. Intensive physical activity and unsuitable nutrition results in the glycogen reserves rapidly becoming exhausted. The preparations according to the invention are also suitable for increasing glycogen resynthesis. Since the resynthesis of the muscle glycogen takes several hours, it is advisable in this type of administration to take the preparations on the day before physical activity, preferably after the evening meal.

Surprisingly, however, it was moreover observed that the preparations obtainable according to the invention, unlike the nutritional constituents needed for glycogen synthesis, are also efficacious if they are consumed a short time before or during physical stress. It is suspected that this action is likewise based on a stimulation of the anaerobic muscular energy metabolism and the preparations can replace the muscle glycogen with respect to its oxygen-sparing action. In this function, the preparations obtainable according to the invention are not replaceable by nutrients such as carbohydrates or proteins.

Furthermore, it was found that the efficacious amount of the first component in the preparations obtainable according to the invention can be further decreased if they are combined with polymeric carrier substances, in particular pharmaceutically acceptable gelling agents such as gellable polymeric carbohydrates (e.g. pectin or agar-agar) or gellable proteins (e.g. gelatin).

The preparations obtainable according to the invention thus permit a significant decrease in the oxygen consumption during physical work and thus also a marked improvement in the efficiency, independently of whether they are taken immediately before or during the physical work or several hours previously. They can be taken as such or employed as a nutritional additive, and they are also suitable, in particular in the case of the low-dose, gelling agent-containing preparations, as pharmaceutical specialities.

According to a preferred aspect, the preparations obtainable according to the invention can contain as a first component D-glucose, D-maltose, a glucogenic amine, a glucogenic amino acid or one which can be metabolized via glyoxylate or a dipeptide or pharmaceutically acceptable salt of such an amino acid and are gelled using a gelling agent. The gelling agent used is preferably a gellable polymeric carbohydrate, in particular agar-agar or pectin, when the first component is D-glucose or D-maltose, or a gellable protein, in particular gelatin, when the first component is a glucogenic amino acid or one which can be metabolized via glyoxylate or a dipeptide or pharmaceutically acceptable salt of such an amino acid.

It was furthermore found that the action of the preparations can often be further increased if they additionally contain pyridoxine (vitamin B₆) or a pharmaceutically acceptable pyridoxine salt such as pyridoxine hydrochloride, ascorbic acid (vitamin C) or a pharmaceutically acceptable ascorbate, such as sodium ascorbate, and/or biotin (vitamin H).

As a rule, the preparations according to the invention contain thiamine or a pharmaceutically acceptable thiamine salt in a dose unit of at least about 5 mg, preferably at least about 10 mg, if no gelling agent is used, or in a dose unit of at least about 0.5 mg, preferably at least about 1 mg, and more preferably at least 2 mg, if a gelling agent is used, and/or a combination of folic acid in a dose unit of at least about 0.1 mg, preferably at least about 0.2 mg, and more preferably at least about 0.3 mg, together with cyanocobalamin in a dose unit of at least about 1 μg, preferably at least about 3 μg. The upper limits of the dose amounts which can be used according to the invention are not critical; in general, however, at most about 1000 mg (preferably at most about 500 mg and more preferably at most about 100 mg) of thiamine or pharmaceutically acceptable thiamine salt, at most about 20 mg of folic acid and at most about 150 μg of cyanocobalamin per dose unit are used. The vitamin dose used according to the invention is thus often markedly above the recommended daily requirement, which is about 1-2 mg for thiamine, about 0.1-0.2 mg for folic acid and about 1 μg for cyanocobalamin.

The dose amounts of the first component of the preparation according to the invention can vary within relatively wide limits, depending on the nature of the component, depending on the desired duration of action and depending on whether a gelling agent is employed or not, it being possible to say as a rough reference point that amino acids and glucogenic amines are comparable with D-glucose in their activity, while ethanol is active even in about 5-times lower doses and D-maltose and dipeptides are active even in about 10- to 50-times lower doses and the activity can be additionally increased by combination with a pharmaceutically acceptable gelling agent and/or other vitamins. The upper limit of the dose ranges of these substances is likewise not critical; in general, however, not more than about 30 g, in the case of ethanol preferably not more than about 10 ml, are used per dose unit. Preferred lower limits and dose ranges of the individual components and other aspects result from the following explanations re preferred embodiments of the present invention.

According to a first embodiment, the preparation according to the invention can preferably contain as a first component D-glucose, D-maltose, a glucogenic amine, a glucogenic amino acid or one which can be metabolized via glyoxylate or a dipeptide or pharmaceutically acceptable salt of such an amino acid and as a second component thiamine or a pharmaceutically acceptable thiamine salt. Preferably, such preparations can contain D-glucose, D-maltose, a glucogenic amine, a glucogenic amino acid or one which can be metabolized via glyoxylate or a pharmaceutically acceptable salt of such an amino acid in a dose unit of at least about 1 g (or in particular in the case of D-maltose or dipeptides of a glucogenic amino acid or one which can be metabolized via glyoxylate even in smaller amounts) and thiamine or a pharmaceutically acceptable thiamine salt in a dose unit of at least about 10 mg, if no gelling agent is employed. If a gelling agent is used, however, even considerably lower minimum amounts of about 100 mg of D-glucose, about 10 mg or 100 mg of D-maltose, about 100 mg of glucogenic amine or about 1 mg of a glucogenic amino acid or one which can be metabolized via glyoxylate or of a dipeptide or pharmaceutically acceptable salt of such an amino acid and about 1 mg of thiamine or pharmaceutically acceptable thiamine salt are sufficient. Dose amounts are particularly preferred which are between the minimum amounts mentioned and an upper limit, which in the case of component (a) is about 30 g and in the case of component (b) about 1000 mg. Usually, however, component (b) is employed in dose amounts of at most about 200 mg and dipeptides are employed in dose amounts of at most about 1 g.

The stimulation of the glycogen synthesis and the oxygen-sparing or efficiency-increasing action on taking briefly before or during physical stress is based on two different mechanisms. On the one hand, the muscular glycogen synthesis is stimulated in the same way as by easily digestible carbohydrates and proteins, this becoming clear only on the day afterwards through utilization of the glycogen for energy production; on the other hand, the efficiency can also be immediately affected if, for example, glucose and vitamin B₁ are taken together immediately before or during physical activity. It is detectable by a reduction of the heart rate with the condition that the muscular glycogen has been broken down previously by physical stress. If glycogen reserves are still present in the muscles, the oxygen-sparing effects are additive in the sense that the glycogen reserves for the time being escape breakdown, until the action of the preparation taken immediately before stress is exhausted, the duration of action being correspondingly prolonged.

The action of the combination preparation, comprising D-glucose, D-maltose, a glucogenic amine, a glucogenic amino acid or one which can be metabolized via glyoxylate or a dipeptide or pharmaceutically acceptable salt of such an amino acid together with vitamin B₁, can be prolonged several times for both indications, i.e. the stimulation of glycogen synthesis and the immediate action on the oxygen requirement, if high doses of vitamin C (ascorbic acid) and/or vitamin B₁₂ (cyanocobalamin) are additionally taken. Thus a total duration of action results which exceeds that of the most active nutritional constituents by several times. A prerequisite for the optimum activity is the joint taking of all components in a balanced ratio. Preferably, the combination preparations mentioned can therefore additionally contain ascorbic acid or a pharmaceutically acceptable ascorbate in a dose unit of at least about 5 mg, in particular about 25 to 1000 mg, and/or cyanocobalamin in a dose unit of at least about 1 μg, preferably about 3 to 150 μg.

The mode of action of this combination preparation can be explained by the functions of the glucose and the vitamins in the metabolism. Vitamin B₁ stimulates the pentose phosphate cycle and as a result of the hydrogen thus released makes possible the resynthesis of phosphoenol pyruvate from pyruvate without the participation of molecular oxygen. Vitamin B₁₂ stimulates the formation of oxaloacetate from pyruvate, which is needed as an intermediate for the synthesis of phosphoenol pyruvate from pyruvate. As an antioxidant, vitamin C protects the glucose during the absorption by the gastric mucous membrane and its transport to the site of action before autoxidation. Glucose, taken as a nutritional constituent, is an energy carrier, 1 g glucose corresponding to about 4 kcal. The oxygen-sparing action of the glucose as a constituent of the combination preparation corresponds to an amount of energy which exceeds this value by several times, from which it is evident that it has a catalytic function which, however, is only expressed when it is taken simultaneously with vitamin B₁ or a combination of vitamin B₁ with vitamin C and/or vitamin B₁₂. The vitamins, for their part, are inactive without addition of glucose or glucose-producing substance. Obviously, only the joint administration causes a stimulation of metabolic processes which are involved in glycogen formation or in anaerobic energy production.

D-Maltose can replace the D-glucose in the preparation according to the invention and is even active in considerably lower amounts. Other types of sugars such as sucrose and fructose prove ineffective, however, for the stimulation of glycogen synthesis and of the anaerobic glucose breakdown in the muscle, even if they are taken together with vitamin B₁ and vitamin C, although these sugars can be converted into glucose in the intermediate metabolism. The different mode of action of sucrose and fructose in comparison with glucose is obviously caused by the fact that the glucose, together with the vitamins, is only active if all components are absorbed together by the gastric mucous membrane immediately after taking. The conversion of the sucrose or fructose to glucose, however, only takes place in the liver if they have been absorbed from the small intestine.

It has likewise been found that fruits or fruit preparations can be employed in the preparation according to the invention instead of pure D-glucose and that in principle all fruits and fruit preparations containing natural glucose are active, it being possible for the activity to vary within certain limits, however, depending on the nature of the fruit and production of the fruit preparations. Preparations of oranges containing concentrated pulp and of dried fruit such as figs, bananas, grapes and pears have proved to be particularly active. Apples are especially suitable in the form of an apple sauce, apples from conventional unfertilized high-stem crops preferably being used. Natural sugar from fruit consists of a mixture of approximately equal parts of glucose, fructose and sucrose, depending on the nature of the fruit, in a slightly variable ratio. It has been shown that clear fruit juices, e.g. from oranges, apples or grapes, are less suitable for enrichment with vitamins, which is why preparations are preferably used which contain fruit flesh. The absorption of the glucose through the gastric mucous membrane is presumably facilitated by selective binding of the glucose to the fruit flesh.

In addition to glucose and maltose, glucogenic amino acids, such as L-alanine, L-serine, L-cysteine, L-cystine, L-glutamic acid, L-aspartic acid, L-arginine, L-ornithine, L-threonine, L-valine, L-isoleucine, L-proline, L-oxyproline, L-tryptophan, L-tyrosine, L-phenylalanine, L-methionine and L-histidine, amino acids which can be converted into glyoxylate, such as glycine, L-serine and L-glutamic acid, dipeptides of glucogenic amino acids or those which can be converted into glyoxylate, such as H-Gly-Gly-OH, H-Ser-Ser-OH and H-Tyr-Tyr-OH, pharmaceutically acceptable salts of glucogenic amino acids or those which can be converted into glyoxylate, such as L-monosodium glutamate and L-monosodium aspartate, as well as glucogenic amines, such as L-glutamine and L-asparagine, are also suitable for the production of active preparations. Glucogenic amino acids are naturally absorbed as a constituent of the nutritive proteins and can be converted into glucose in the intermediate metabolism. Of 14 regularly taken glucogenic amino acids, L-alanine, L-aspartic acid, L-glutamic acid and L-serine especially have proved particularly suitable with respect to their activity and accessibility for the production of active preparations. All four amino acids can be activated by addition of vitamin B₁ without great differences in the activity and, for example, to the same extent as glucose.

The addition of vitamin C to a glucogenic amino acid and vitamin B₁ results in a marked further improvement in the activity, however, only in the case of L-aspartic acid, L-aspartate, L-phenylalanine, L-tyrosine and L-tryptophan. The same also applies to additional vitamin B₁₂. An outstanding action is thus achieved by a combination of one of these amino acids with vitamin B₁ and vitamin C and/or vitamin B₁₂, where these can preferably be employed in the dose amounts indicated above and an additional increase can also be achieved by addition of pyridoxine (vitamin B₆) or a pharmaceutically acceptable pyridoxine salt, such as pyridoxine hydrochloride. Preferably, the latter is employed in a dose unit of at least about 20 mg, in particular about 50 to 1000 mg (i.e. in an amount which corresponds to several times the recommended daily requirement of 2-4 mg), if no gelling agent is used. If a gelling agent is used, however, even dose amounts of at least about 1 mg, preferably at least about 2 mg, are usually sufficient.

The four preferably utilizable glucogenic amino acids mentioned or their salts can be differently utilized in the intermediate metabolism. Specifically, several metabolic pathways are open, even those which do not lead to glucose, so that competition situations can arise. The outstanding action of L-aspartic acid or of an L-aspartate combined with four vitamins is obviously caused by the fact that it is immediately converted into oxaloacetic acid and this reaction is catalysed by the vitamin B₆.

The D-glucose, D-maltose, a glucogenic amine, a glucogenic amino acid or one which can be metabolized via glyoxylate or a dipeptide or pharmaceutically acceptable salt of such an amino acid-containing preparation according to the previously described embodiment can be present in solid form, together with suitable, pharmaceutically acceptable vehicles, diluents or excipients, or in the form of an aqueous solution or suspension, where in the latter case the first components can preferably be a fruit juice or fruit concentrate, comprising natural D-glucose in efficacious amount.

In addition to the glucose formation, L-serine and L-glutamic acid can be converted into glyoxylate. This metabolism is also open to the amino acid glycine. Via glyoxylate formation, an additional stimulation of muscular glycogen synthesis and anaerobic energy formation is made possible, if by means of addition of high doses of folic acid and vitamin B₁₂ the metabolic utilization of glyoxylate to formyl and formate formation is stimulated and ATP is thus formed anaerobically. Combination preparations of glycine, L-glutamic acid and L-serine with high doses of folic acid and vitamin B₁₂ show a similar activity to those which contain L-serine or L-glutamic acid and vitamin B₁.

According to a further preferred embodiment, the preparation obtainable according to the invention can therefore preferably contain as a first component glycine, L-serine, L-glutamic acid or a dipeptide (e.g. H-Gly-Gly-OH or H-Ser-Ser-OH) or pharmaceutically acceptable salt (e.g. L-sodium glutamate) thereof and as a second component a combination of folic acid and cyanocobalamin. Preferably, these preparations can contain glycine, L-serine, L-glutamic acid or a dipeptide or pharmaceutically acceptable salt thereof in a dose unit of at least 1 g, in particular about 5 to 30 g, or in the case of dipeptides even markedly less, if no gelling agent is used.

In the presence of a gelling agent, however, as a rule even dose amounts of at least about 1 mg, preferably at least about 2 mg, are sufficient. The dose amounts of the second component can be at least about 0.1 mg, preferably at least about 0.2 mg, in particular about 1 to 20 mg, for folic acid and at least about 1 μg, in particular about 3 to 150 μg, for cyanocobalamin. These preparations can likewise be present in solid form, together with suitable, pharmaceutically acceptable vehicles, diluents or excipients, or in the form of an aqueous solution or suspension.

According to conventional teaching, ethanol is broken down in the liver to acetate and this is used for energy utilization or for the synthesis of fats. Surprisingly, it has been shown that catalytic amounts of ethanol, taken together with efficacious doses of biotin (vitamin H) and vitamin B₁, can stimulate both the muscular glycogen synthesis and anaerobic metabolic reactions of the muscles for energy utilization. The action of such preparations takes place immediately after taking, so that it is possible to detect that it occurs without involvement of the liver. A plausible explanation for this results from the idea that biotin stimulates the carboxylation of pyruvate and thus oxaloacetate synthesis, vitamin B₁ makes the formation of phosphoenol pyruvate from oxaloacetate possible indirectly via the pentose phosphate cycle and the acetate formed from ethanol together with the oxaloacetate stimulates the citrate cycle and thus energy for phosphoenol pyruvate synthesis is available via the synthesis of GTP (guanosine triphosphate).

According to a further preferred embodiment, the preparation obtainable according to the invention can therefore contain ethanol as a first component, thiamine or a pharmaceutically acceptable thiamine salt as a second component and preferably biotin as a further component, where ethanol can preferably be present in a dose unit of at least about 0.2 g (for example about 1 to 10 ml, 0.25-1 g as a rule being sufficient), thiamine or a pharmaceutically acceptable thiamine salt in a dose unit of at least about 5 mg (in particular about 10 to 1000 mg) and biotin in a dose unit of at least about 0.1 mg (in particular about 0.3 to 10 mg), if used in the absence of a gelling agent. If the preparation is gelled utilizing a gelling agent, in particular a gellable polymeric carbohydrate such as pectin or agar-agar or a gellable protein such as gelatin, the suitable doses of ethanol, thiamine or thiamine salt and biotin can however be decreased. In gelled preparations it is usually sufficient to use ethanol in a dose unit of at least about 10 mg, preferably at least about 25 mg, thiamine or a pharmaceutically acceptable thiamine salt in a dose unit of at least about 0.5 mg, preferably at least about 1 mg, and biotin, if present, in a dose unit of at least about 25 μg, preferably at least about 50 μg. Preferably, the preparations comprising ethanol and thiamine or thiamine salt can be present in the form of aqueous solutions.

Muscular glycogen synthesis and anaerobic energy formation can also be increased in combinations of catalytic amounts of ethanol with high doses of folic acid and vitamin B₁₂. The action of such preparations excels that of the combination of ethanol, biotin and vitamin B₁ and is presumably based on a hitherto unknown conversion of the ethanol into glyoxal in the musculature.

According to a further embodiment, the preparation obtainable according to the invention can therefore preferably contain ethanol and—as a second component—a combination of folic acid and cyanocobalamin in efficacious amounts, ethanol preferably being used in a dose unit of at least about 0.2 g (for example about 1 to 10 ml, 0.25-1 g as a rule being sufficient), folic acid in a dose unit of at least about 0.2 mg (in particular about 0.5 to 20 mg) and cyanocobalamin in a dose unit of at least about 1 μg (in particular about 3 to 150 μg), if used in the absence of a gelling agent. If the preparation is gelled utilizing a gelling agent, in particular a gellable polymeric carbohydrate such as pectin or agar-agar or a gellable protein such as gelatin, the suitable doses of ethanol, folic acid and cyanocobalamin can be decreased. In gelled preparations it is usually sufficient to use ethanol in a dose unit of at least about 10 mg, preferably at least about 25 mg, folic acid in a dose unit of at least about 0.1 mg, preferably at least about 0.2 mg, and cyanocobalamin in a dose unit of at least about 1 μg, preferably about 3 to 150 μg. These preparations can preferably also be administered as aqueous solutions.

The glucose, as a nutritional constituent, is subject to the activity of insulin, so that their action is prevented by therapeutic doses of insulin, sulphonylurea derivatives having antidiabetic activity, glucocorticoids, chlorprothixene or thioxanthene (i.e. active compounds which inhibit the breakdown or the synthesis of glycogen) . The main metabolic action of insulin is the regulation of the intermediary glucose metabolism. The therapeutic effect of antidiabetic sulphonylurea derivatives, such as chlorpropamide, glibornuride, glibencalmide, tolbutamide and acetohexamide, is attributed to the stimulation of insulin secretion from the pancreas. In this context insulin increases the formation of glycogen in liver and muscles and inhibits the degradation of glucose. The metabolic utilization of liver glycogen is distinctly different from that of muscle glycogen. The glycogen stored in the liver is extremely labile. After fasting overnight, the glycogen content of the liver is reduced by multiple. It is rapidly resynthesized by carbohydrate-rich diets. Muscle glycogen is not particularly affected, neither by fasting nor by carbohydrate-rich diets, when at rest. It is rapidly degraded by heavy exercise. The resynthesis of muscular glycogen after degradation requires carbohydrate-rich diets and is greatly stimulated by previous complete depletion of by heavy exercise. Glucocorticoids, such as dexamethasone, prednisolone, prednisone and cortisol, act in the metabolism as antagonists of insulin. Their overall effects on metabolism are almost opposite of those of insulin. In the muscles they inhibit the utilization of glucose for the formation of glycogen.

However, the efficacy of preparations which as a first component contain a glucogenic amino acid or one which can be metabolized via glyoxylate, a dipeptide or pharmaceutically acceptable salt of such an amino acid or ethanol and, as a second component, thiamine or a pharmaceutically acceptable thiamine salt, preferably together with biotin, or a combination of folic acid and cyanocobalamin, is not adversely affected by insulin, sulphonylurea derivatives having antidiabetic activity, glucocorticoids, chlorprothixenes and thioxanthenes. Moreover, it has surprisingly been found that an oxygen-sparing action is still achieved, if the first component (a) is D-glucose, D-maltose or a glucogenic amine, however with the proviso that, in the case of the first component being D-glucose and the medicament being an antidiabetic sulphonylurea derivative, the combination is ingested before sleep, the evening before the physical exercise. The present methods and preparations thus permit a decrease in the oxygen consumption even on administration of such agents and are therefore suitable for the prevention of side effects of such agents, the therapeutic activity of the pharmaceutical active compounds mentioned not being affected.

A preferred aspect of the present invention therefore relates to preparations comprising a first component (a) and a second component (b), as defined hereinbefore, together with a medicament selected from the group consisting of insulin, antidiabetic sulphonylurea derivatives, glucocorticoids, chlorprothixenes and thioxanthenes, and to a method comprising administering simultaneously to an animal including human efficacious amounts of said components (a) and (b) and of said medicament. Administration can be effected by separate but simultaneous ingestion of the components and medicament or by ingestion of said preparation comprising the components and medicament.

According to a preferred embodiment, the preparations obtainable according to the invention, which as a first component contain a glucogenic amino acid or one which can be metabolized via glyoxylate, a dipeptide or pharmaceutically acceptable salt of such an amino acid or ethanol, can therefore contain as a further component insulin, a sulphonylurea derivative having antidiabetic activity, a glucocorticoid, chlorprothixene or thioxanthene in a therapeutically efficacious amount. Preferably, the medicament can be a glucocorticoid and the first component can be ethanol or a dipeptide of an amino acid which can be metabolized via glyoxylate in which case administration can preferably be effected shortly before or during the physical work. If thiamine or a pharmaceutically acceptable thiamine salt is used as a second component in such a preparation, the preparation can preferably additionally contain an efficacious amount of biotin. It is particularly preferred, however, in this application to use the dipeptide of an amino acid which can be metabolized via glyoxylate, preferably the dipeptide of glycine, L-serine or L-glutamic acid, as a first component, and a combination of folic acid and cyanocobalamin as a second component and to gel the preparation using a gellable protein, preferably gelatin. A further preferred embodiment concerns simultaneous administration of efficacious amounts of (a) D-glucose or D-maltose, (b) thiamine or a pharmaceutically acceptable thiamine salt or a combination of folio acid and cyanocobalamin, and (c) a glucocorticoid, as well as preparations comprising efficacious amounts of (a) D-glucose or D-maltose, (b) thiamine or a pharmaceutically acceptable thiamine salt, and (c) a glucocorticoid; in this embodiment, administration is preferably effected shortly (preferably within one hour or more preferably within 15 or 30 minutes) before or during the physical work. With respect to suitable manners of administration, administration forms, use of a gelling agent and preferred dose ranges, reference is made to the above details.

Most surprisingly, it has further been found that antidiabetic sulphonylurea derivatives reduce oxygen consumption and heat production, if ingested the evening before the physical exercise, and that combined ingestion together with a glucose preparation of the present invention is suitable to improve the effect of the latter in a synergistic manner. A particularly preferred aspect of the invention therefore relates to a method of decreasing oxygen consumption in animal including human during physical work which comprises simultaneous administration of efficacious amounts of (a) D-glucose, (b) thiamine or a pharmaceutically acceptable thiamine derivative, and (c) an antidiabetic sulphonylurea derivative on the day (preferably in the evening) before the physical work. Preferably, simultaneous administration may be effected by ingesting a preparation comprising efficacious amounts of components (a), (b) and (c) or by ingesting simultaneously an efficacious amount of an antidiabetic sulphonylurea derivative and a preparation comprising efficacious amounts of components (a) and (b). With respect to suitable manners of administration, administration forms, use of a gelling agent and preferred dose ranges, reference is made to the above details. However, the sulphonylurea derivatives are effective in this application at much lower doses than in their use as antidiabetics. Typically, the suitable doses are about one tenth, or even less, of the recommended doses as antidiabetics.

As already explained above, the preparations obtainable according to the invention, in particular if they are present in the form of an aqueous solution or suspension, can contain a pharmaceutically acceptable gelling agent, in particular a gellable polymeric carbohydrate or a gellable protein, such as pectin, agar-agar or gelatin. In particular, it was found that the efficacious amount of D-glucose, D-maltose, ethanol, glucogenic amine, glucogenic amino acid or amino acid which can be metabolized via glyoxylate or of dipeptide or pharmaceutically acceptable salt of such an amino acid and in some cases also the efficacious amount of vitamins can be further decreased if this is present in combination with such a polymeric carrier. The amount of gelling agent is not critical; for example, it can be employed in an amount of about 5 to 200 g per 1 of water. Gelling can be carried out in a manner known per se by dissolving the components in water, heating and cooling.

Preparations obtainable according to the invention, which are present in the form of aqueous solutions or suspensions, e.g. as a beverage solution, can contain the first and second components in a total concentration of, for example, about 1 to 50% by weight. The concentration, however, is not critical.

Preparations obtainable according to the invention, which can be present in solid form, e.g. as an effervescent powder, granules or tablet, can, if desired, contain suitable, pharmaceutically acceptable vehicles, diluents or excipients such as sodium hydrogencarbonate, citric acid, mannitol, talc, maize starch, glyceryl monostearate, food colorants, aromatic substances and the like.

According to the two manners of administration, an oxygen-sparing or performance-increasing action can be achieved either shortly after taking or—by increasing the glycogen synthesis—on the following day using the preparations according to the invention, it being irrelevant to the efficacy whether the active components are present in a preparation together or are taken individually; it is only crucial that the active components are taken simultaneously. The present invention therefore makes possible a method for decreasing the oxygen consumption during physical work, which comprises taking the preparation obtainable according to the invention or the active components individually, but simultaneously, either on the day before physical work, preferably after the evening meal, or shortly before or during physical stress, preferably not longer than about 30 minutes before physical stress.

The taking of the preparation obtainable according to the invention or of the active components is indicated, for example

in the case of intensive physical stress as a result of athletic or occupational activity,

in the case of lung, circulation and heart disorders to improve the physical performance or to prevent dyspnoea,

during stays in high regions,

in the case of metabolic disorders caused by disturbances of the carbohydrate metabolism, e.g. in diabetes mellitus,

for the elimination of side effects of medicinal therapy, which are caused by adverse effects on the carbohydrate metabolism, e.g. by simultaneous administration with glucocorticoids,

for the improvement of physical performance during slimming cures and

for use in animal nutrition and veterinary medicine, in particular in the case of mammals, for analogous purposes, e.g. for increasing the performance in racing.

The preparations obtainable according to the invention are in principle also suitable for use in animal nutrition, in particular in the case of mammals, for the purpose of improving the meat quality and the physical efficiency.

The present invention furthermore relates to a preparation for decreasing the oxygen consumption during physical work, which contains (a) an efficacious amount of ethanol as a first component and (b) an efficacious amount of a combination of folic acid and cyanocobalamin or an efficacious amount of thiamine or of a pharmaceutically acceptable thiamine salt, preferably in combination with biotin, as a second component. The preparation preferably contains either ethanol in a dose unit of at least 0.2 g, folic acid in a dose unit of at least 0.2 mg and cyanocobalamin in a dose unit of at least 1 μg or ethanol in a dose unit of at least 0.2 g, thiamine or a pharmaceutically acceptable thiamine salt in a dose unit of at least 5 mg and biotin in a dose unit of at least 0.1 mg. As mentioned above, the preparation can preferably be gelled utilizing a gelling agent, whereby the suitable doses can be further decreased.

The invention likewise relates to a preparation for decreasing the oxygen consumption during physical work, which contains the following components:

(a) an efficacious amount of D-glucose, D-maltose, ethanol, of a glucogenic amine, of a glucogenic amino acid or one which can be metabolized via glyoxylate or of a dipeptide or pharmaceutically acceptable salt of such an amino acid as a first component,

(b) an efficacious amount of thiamine, of a pharmaceutically acceptable thiamine salt or of a combination of folic acid and cyanocobalamin as a second component, with the proviso that the second component is thiamine or a pharmaceutically acceptable thiamine salt when the first component is D-glucose, D-maltose, a glucogenic amine, a glucogenic amino acid which is not metabolizable via glyoxylate, or a dipeptide or pharmaceutically acceptable salt of such an amino acid is present as the first component, and

(c) a gelling agent as a further component, the gelling agent preferably being a gellable polymeric carbohydrate when the first component is D-glucose, D-maltose, ethanol or a glucogenic amine, or a gellable protein when the first component is ethanol or a glucogenic amino acid or one which can be metabolized via glyoxylate or a dipeptide or pharmaceutically acceptable salt of such an amino acid.

According to a first embodiment, it can preferably contain an efficacious amount of D-glucose or D-maltose, an efficacious amount of thiamine or of a pharmaceutically acceptable thiamine salt, a gellable polymeric carbohydrate, in particular agar-agar or pectin, and, if desired, additionally ascorbic acid or a pharmaceutically acceptable ascorbate and/or cyanocobalamin and/or pyridoxine or a pharmaceutically acceptable pyridoxine salt in efficacious amount. According to a second embodiment, the preparation can preferably contain an efficacious amount of a glucogenic amino acid or one which can be metabolized via glyoxylate or of a dipeptide or pharmaceutically acceptable salt of such an amino acid, an efficacious amount of thiamine or of a pharmaceutically acceptable thiamine salt, a gellable protein, in particular gelatin, and, if desired, additionally ascorbic acid or a pharmaceutically acceptable ascorbate and/or cyanocobalamin and/or pyridoxine or a pharmaceutically acceptable pyridoxine salt in efficacious amount. According to a third embodiment, the preparation preferably contains an efficacious amount of an amino acid which can be metabolized via glyoxylate or of a dipeptide or pharmaceutically acceptable salt of such an amino acid, efficacious amounts of folic acid and cyanocobalamin and a gellable protein, in particular gelatin; in this embodiment the use of the dipeptides H-Gly-Gly-OH, H-Ser-Ser-OH and H-Glu-Glu-OH is particularly preferred. According to a further embodiment, the preparation can preferably be a gelled ethanol-containing composition, as disclosed above.

The invention furthermore relates to a pharmaceutical preparation containing insulin, a sulphonylurea derivative having antidiabetic activity, a glucocorticoid, chlorprothixene or thioxanthene in therapeutically efficacious amount, which contains, for avoiding or decreasing side effects (a) an efficacious amount of a glucogenic amino acid or one which can be metabolized via glyoxylate or of a dipeptide or pharmaceutically acceptable salt of such an amino acid, preferably an efficacious amount of H-Gly-Gly-OH, H-Ser-Ser-OH and/or H-Glu-Glu-OH, (b) an efficacious amount of thiamine, a pharmaceutically acceptable thiamine salt or a combination of folic acid and cyanocobalamin and (c) a gellable protein.

The preparation, the mode of action and the preferred dose ranges of the preparations according to the invention follow from the preceding explanations.

The invention is illustrated further by the following examples. Glucose and maltose were in each case used in the D form, amino acids in the L form.

### EXAMPLE 1

500 g of L-glutamic acid monosodium salt, 10 g of thiamine mononitrate, 100 g of sodium bicarbonate and 100 g of citric acid are intimately mixed in a dry room. The effervescent powder obtained is dispensed into sachets of 7 g each. A dose of 7 g produces a slightly effervescent solution on sprinkling into about 30-50 ml of water.

### EXAMPLE 2

500 g of D-glucose (finely crystalline), 10 g of thiamine nitrate and 10 g of ascorbic acid are granulated in a 5% strength gelatin solution in alcohol water (e.g. Spiritus gelatinae Ph.) as adhesive solution. 100 g of sodium bicarbonate and 100 g of citric acid are admixed to the dried and finely sieved granules. The effervescent granules obtained are divided into portions of 7 g.

### EXAMPLE 3

The effervescent granules obtained according to Example 2 are pressed to give tablets of 3.5 g after addition of 90 g of talc as a lubricant and 100 g of maize starch as a disintegrant. 1% glyceryl monostearate (monostearin) can be added to the tabletting mixture as a disintegration accelerator.

### EXAMPLE 4

5 g of maltose, 10 g of pectin, 1 g of thiamine hydrochloride, 1 g of ascorbic acid and 150 μg of cyanocobalamin are dissolved with heating in 1 l of water and cooled for gelling. 10 g of the product correspond to an oral dose.

### EXAMPLE 5

2 g of H-Gly-Gly-OH, 2 g of gelatin and 1 g of thiamine hydrochloride are added to 100 ml of water, heated after 5 minutes and then cooled for gelling. 1 g of the product corresponds to an oral dose.

### EXAMPLE 6

100 mg of H-Ser-Ser-OH, 200 mg of gelatin, 10 mg of folic acid and 30 μg of cyanocobalamin are added to 10 ml of water, heated after 5 minutes and then cooled for gelling. 250 mg of the product correspond to an oral dose.

### EXAMPLE 7

10 g of ethanol, 250 mg of thiamine hydrochloride and 10 mg of biotin are dissolved in 100 ml of water. 4 ml of the solution correspond to an oral dose.

### EXAMPLES 8-32

By mixing the components, the following fruit preparations, dry mixtures, solutions or suspensions are prepared. The quantitative data in each case relate to an oral dose.

| | | |
|---|---|---|
| Ex. 8: | 10 | g of glucose |
| | 100 | mg of thiamine |
| | 100 | ml of water |
| Ex. 9: | 10 | g of glucose |
| | 100 | mg of thiamine |
| | 100 | mg of ascorbic acid |
| | 100 | ml of water |
| Ex. 10: | 10 | g of glucose |
| | 100 | mg of thiamine |
| | 100 | mg of ascorbic acid |
| | 15 | μg of cyanocobalamin |
| | 100 | ml of water |
| Ex. 11: | 10 | g of dried fig |
| | 50 | mg of thiamine |
| | 5 | g of water |
| Ex. 12: | 10 | g of dried fig |
| | 50 | mg of thiamine |
| | 50 | mg of ascorbic acid |
| | 5 | g of water |
| Ex. 13: | 10 | g of dried fig |
| | 50 | mg of thiamine |
| | 50 | mg of ascorbic acid |
| | 15 | μg of cyanocobalamin |
| | 5 | g of water |
| Ex. 14: | 100 | g of orange juice with doubled pulp |
| | | concentration |
| | 50 | mg of thiamine |
| | 50 | mg of ascorbic acid |
| | 15 | μg of cyanocobalamin |
| Ex. 15: | 50 | g of apple sauce (high-stem fruit from |
| | | biological cultivation) |
| | 25 | mg of thiamine |
| | 25 | mg of ascorbic acid |
| Ex. 16: | 50 | g of apple sauce (high-stem fruit from |
| | | biological cultivation) |
| | 25 | mg of thiamine |
| | 25 | mg of ascorbic acid |
| | 4 | μg of cyanocobalamin |
| Ex. 17: | 50 | g of banana flesh, homogenized with |
| | | 10 ml of water |
| | 25 | mg of thiamine |
| | 25 | mg of ascorbic acid |
| Ex. 18: | 50 | g of banana flesh, homogenized with |
| | | 10 ml of water |
| | 25 | mg of thiamine |
| | 25 | mg of ascorbic acid |
| | 4 | μg of cyanocobalamin |
| Ex. 19: | 10 | g of alanine |
| | 100 | mg of thiamine |
| | 100 | ml of water |
| Ex. 20: | 10 | g of serine |
| | 100 | mg of thiamine |
| | 100 | mg of pyridoxine |
| | 100 | ml of water |
| Ex. 21: | 10 | g of monosodium glutamate |
| | 100 | mg of thiamine |
| | 100 | ml of water |
| Ex. 22: | 1 | g of monosodium aspartate |
| | 50 | mg of thiamine |
| | 50 | mg of ascorbic acid |
| | 50 | mg of pyridoxine |
| | 15 | μg of cyanocobalamin |
| Ex. 23: | 10 | g of serine |
| | 5 | mg of folic acid |
| | 15 | μg of cyanocobalamin |
| | 100 | ml of water |
| Ex. 24: | 10 | g of monosodium glutamate |
| | 5 | g of folic acid |
| | 15 | μg of cyanocobalamin |
| | 100 | ml of water |
| Ex. 25: | 1 | g of ethanol |
| | 100 | mg of thiamine |
| | 2.5 | mg of biotin |
| | 20 | ml of water |
| Ex. 26: | 1 | ml of ethanol |
| | 10 | mg of thiamine |
| | 0.3 | mg of biotin |
| | 2.5 | mg of glibenclamide |
| | 1 | ml of water |
| Ex. 27: | 1 | ml of ethanol |
| | 10 | mg of thiamine |
| | 0.3 | mg of biotin |
| | 12.5 | mg of glibornuride |
| | 1 | ml of water |
| Ex. 28: | 1 | ml of ethanol |
| | 10 | mg of thiamine |
| | 0.3 | mg of biotin |
| | 5 | mg of prednisolone |
| | 1 | ml of water |
| Ex. 29: | 1 | g of ethanol |
| | 5 | g of folic acid |
| | 15 | μg of cyanocobalamin |
| | 20 | ml of water |
| Ex. 30: | 1 | ml of ethanol |
| | 0.5 | mg of folic acid |
| | 3 | μg of cyanocobalamin |
| | 2.5 | mg of glibenclamide |
| | 1 | ml of water |
| Ex. 31: | 1 | ml of ethanol |
| | 0.5 | mg of folic acid |
| | 3 | μg of cyanocobalamin |
| | 12.5 | mg of glibornuride |
| | 1 | ml of water |
| Ex. 32: | 500 | mg of ethanol |
| | 300 | μg of folic acid |
| | 5 | μg of cyanocobalamin |
| | 10 | ml of water |

### EXAMPLES 33-36

Analogously to Examples 4-6, the following preparations are prepared by dissolving the components in water, heating and then gelling by cooling. The quantitative data in each case relate to an oral dose.

| | | |
|---|---|---|
| Ex. 33: | 200 | mg of glucose |
| | 10 | mg of thiamine |
| | 10 | mg of ascorbic acid |
| | 5 | μg of cyanocobalamin |
| | 100 | mg of agar-agar |
| | 5 | ml of water |
| Ex. 34: | 12 | mg of maltose |
| | 10 | mg of thiamine |
| | 10 | mg of ascorbic acid |
| | 5 | μg of cyanocobalamin |
| | 100 | mg of agar-agar |
| | 5 | ml of water |
| Ex. 35: | 10 | mg of L-tyrosine |
| | 2 | mg of thiamine |
| | 5 | mg of ascorbic acid |
| | 2 | mg of pyridoxine |
| | 2 | μg of cyanocobalamin |
| | 20 | mg of gelatin |
| | 1 | ml of water |
| Ex. 36: | 5 | mg of H-Ser-Ser-OH |
| | 300 | μg of folic acid |
| | 5 | μg of cyanocobalamin |
| | 20 | mg of gelatin |
| | 1 | ml of water |

### EXAMPLE 37

The oxygen absorption can be determined by measuring the heart rate as a function of the mechanical stress. This ergometric method is based on the determination of the oxygen transport volume by the cardiac activity. Since the stroke volume, independently of the intensity of the mechanical work, is essentially constant and the increased requirement for oxygen, caused by increasing the work, results in an increase in the heart rate, the quantity of the transported oxygen can be established by determining the additional heartbeats caused by the work. A 3-stage ergometer test recommended by the World Health Organization (WHO) is used world-wide in physiological research institutes and fitness clubs.

The working muscle utilizes glycogen and fatty acids together to obtain energy. The muscle glycogen is broken down anaerobically. The breakdown of fatty acid is dependent on molecular oxygen. When the glycogen reserves are exhausted and only fats are available, the oxygen requirement and thus also the heart rate are increased. Under constant stress, the heart rate therefore increases slightly in the presence of muscle glycogen immediately after the start of the mechanical stress and remains at a low level as long as glycogen is available. After its exhaustion, a fresh increase takes place to a higher level, which is ascribed to the exclusive utilization of fats. The oxygen transport volume of the heart action can therefore be determined by establishment of the number of heartbeats in two cases of physical stress which are different, but in each case kept constant, as a result of mechanical work (of, for example, at least 5 minutes each) if the utilization of the muscle glycogen for energy supply is eliminated during the test period in favour of the exclusive utilization of fats by completely breaking down the muscle glycogen immediately before the determination process either by physical stress of the musculature involved in the mechanical work or blocking its oxygen-sparing action by administration of an efficacious dose of an antidiabetic agent. The oxygen transport volume can then be calculated from the difference between the mechanical work and the corresponding difference between the number of heartbeats, an efficiency of 20% being used as a basis in the following calculations, with the assumption that a fifth of the oxygen absorbed is used for the mechanical work and the remainder for heat production. The metabolic oxygen requirement for the utilization of 1 kcal is 200 ml.

To determine the oxygen-sparing action of the preparations obtainable according to the invention, a 60 year-old, male subject having a good condition index was exposed on successive days, in each case in the morning, to a constant physical stress of 100 W (60 revolutions per minute on a bicycle ergometer) for 10 minutes. Per heart contraction, an oxygen transport volume of 32 ml was determined beforehand according to the method explained above. The tests were in each case carried out in the fasting state. Before the start of the test, the glycogen reserves were completely broken down by physical stress of 100 W. At the start of the test, i.e. immediately before the physical stress—apart from in the control test—one of the preparations of Examples 32-36 (in the amount indicated there) was administered in each case. The heart rate was measured at one-minute intervals. The values of the heart rate, the number of the heartbeats induced by the physical stress and the oxygen absorption determined are compiled in Table 1.

To maintain a constant mechanical stress, a bicycle ergometer from Ergo-Fit & Co. (Pirmasens, Germany) was used, whose resistance is controllable by means of an eddy current brake. The maintenance of a constant pedal speed was additionally guaranteed by acoustic signals of a metronome from Seiko (Japan). Continuous counting of the heartbeats and recording thereof was carried out by means of a heart rate computer from Polar Electro OY (Kempele, Finland).

TABLE 1

| | | Number of | |
|---|---|---|---|
| | Average heart | heartbeats | |
| | rate | induced by | Oxygen |
| | [heartbeats | work during | absorption |
| Preparation | per min.] | 10 min. | [1/min.] |
| Control | 140 | 796 | 2.55 |
| Ex. 32 | 101 | 412 | 1.32 |
| Ex. 33 | 97 | 373 | 1.12 |
| Ex. 34 | 98 | 381 | 1.22 |
| Ex. 35 | 97 | 372 | 1.12 |
| Ex. 36 | 99 | 404 | 1.29 |

### EXAMPLE 38

Analogously to Example 37, the oxygen-sparing action of the preparations obtainable according to the invention was investigated during simultaneous administration of insulin to a 30 year-old cyclist in a good state of fitness having an oxygen transport volume per heart contraction of 36 ml. At the start of the test, the subject was injected with 5 IU of insulin in each case both in the control test and together with the administration of a preparation in order to block a possible oxygen-sparing action by the muscle glycogen. During the 10-minute test period, a constant physical stress of 175 W (60 revolutions per minute) was maintained. Otherwise, the tests were carried out as described in Example 37. The values obtained are compiled in Table 2.

TABLE 2

| | | Number of | |
|---|---|---|---|
| | Average heart | heartbeats | |
| | rate | induced by | Oxygen |
| | [heartbeats | work during | absorption |
| Preparation | per min.] | 10 min. | [1/min.] |
| Control | 131 | 722 | 2.60 |
| Ex. 32 | 76 | 162 | 0.58 |
| Ex. 36 | 74 | 144 | 0.52 |

### EXAMPLE 39

Analogously to Example 37, the following studies a) to c) were carried out on two male individuals, trained for endurance cycling and kept on a permanently carbohydrate-rich diet; individual A (67 years old, weighing 67 kg) having a maximum oxygen uptake capacity of 3.3 l and an oxygen transport volume per heart beat of 29 ml (corresponding to 0.6 kJ), and individual B (42 years old, weighing 74 kg) having a maximum oxygen uptake capacity of 4.5 l and an oxygen transport volume per heart beat of 36 ml (corresponding to 0.75 kJ) . The following preparations were used, in which the indicated amounts correspond to one dose unit and which were obtained by dissolving or suspending the components in water and gelling the mixture:

Prep. I: 10 mg of D-glucose, 1 mg of thiamine and 10 mg of ascorbic acid heated with 5 mg of agar-agar in 1 ml of water;

Prep. II: 25 mg of ethanol, 1 mg of thiamine and 50 μg of biotin heated with 5 mg of agar-agar in 1 ml of water;

Prep. III: 25 mg of ethanol, 250 μg of folic acid and 5 μg of cyanocobalamin heated with 5 mg of gelatin in 1 ml of water;

Prep. IV: 10 mg of H-Gly-Gly-OH, 300 μg of folic acid and 5 μg of cyanocobalamin heated 5 mg of gelatin in 1 ml of water.

H-Gly-Gly-OH was bought from Novabiochem, Läufelfingen, Switzerland. Insulin was injected subcutaneously, while the other medicaments were ingested alone or together with one of Preparations I-IV.

a) In a first study, insulin or antidiabetic sulphonylurea derivatives were applied, alone or in combination with gelatin and/or Preparation I or II, 15 minutes before the tests. In the tests, individual A was subjected to a workload of 100 W (60 r.p.m.) for 15 minutes (tests A1-A8), and individual B was subjected to a workload of 175 W (60 r.p.m.) for 10 minutes (tests B9-B29). The mean heart rate as well as the heat production and oxygen consumption induced by the workload are summarized in Table 3.

The results show that insulin and antidiabetic sulphonylurea derivatives increase the heat production and oxygen consumption induced by constant physical work loads, and that this effect is prevented by simultaneous intake of Preparation II, whereas gelatin and/or Preparation I are not suitable to prevent the effect if taken shortly before the test.

TABLE 3

| | | Mean | | |
|---|---|---|---|---|
| | | heart | Heat | Oxygen |
| | | rate | production | consumption |
| Test | Dosage | [per min.] | [kJ] | [liter] |
| A1-5 | Control (averages | 116.1 | 505 | 24 |
| | without dosage) | | | |
| A6 | 2.5 mg glibenclamide | 135.4 | 679 | 33 |
| A7 | 2.5 mg glibenclamide | 136.4 | 687 | 33 |
| A8 | 2.5 mg glibenclamide | 137.8 | 700 | 34 |
| B9-14 | Control (averages | 99.5 | 356 | 17 |
| | without dosage) | | | |
| B15 | 5 I.U. insulin | 142.0 | 675 | 32 |
| B16 | 5 I.U. insulin | 144.8 | 696 | 33 |
| B17 | 5 I.U. insulin | 144.8 | 696 | 33 |
| B18 | 5 I.U. insulin + | 142.5 | 679 | 33 |
| | Prep. I | | | |
| B19 | 5 I.U. insulin + | 100.7 | 365 | 18 |
| | Prep. II | | | |
| B20 | 2.5 mg glibenclamide | 147.5 | 724 | 34 |
| B21 | 2.5 mg glibenclamide | 145.1 | 698 | 34 |
| B22 | 100 μg glibenclamide + | 136.1 | 631 | 30 |
| | 5 mg gelatin | | | |
| B23 | 200 μg glibenclamide + | 143.0 | 682 | 33 |
| | 5 mg gelatin | | | |
| B24 | 10 mg chlorpropamide + | 143.7 | 688 | 33 |
| | 5 mg gelatin | | | |
| B25 | 20 mg chlorpropamide + | 144.0 | 691 | 33 |
| | 5 mg gelatin | | | |
| B26 | 100 μg glibenclamide + | 142.0 | 675 | 32 |
| | 5 mg gelatin + Prep. I | | | |
| B27 | 100 μg glibenclamide + | 98.8 | 351 | 17 |
| | 5 mg gelatin + Prep. II | | | |
| B28 | 10 mg chlorpropamide + | 144.1 | 691 | 33 |
| | 5 mg gelatin + Prep. I | | | |
| B29 | 10 mg chlorpropamide + | 97.3 | 340 | 16 |
| | 5 mg gelatin + Prep. II | | | |

b) In a second study, antidiabetic sulphonyl urea derivatives, glucocorticoids, Preparation I and combinations thereof were administered before sleep, the evening before the tests. In the tests, individual A was subjected to a workload of 100 W (60 r.p.m.) for 25 minutes (tests A1-A9), and individual B was subjected to a workload of 175 W (60 r.p.m.) for 45 minutes (tests B10-B33). The mean heart rate as well as the heat production and oxygen consumption induced by the workload are summarized in Table 4.

The results show that—in contrast to the effect found in the preceding study—antidiabetic sulphonylurea derivatives reduce the heat production and oxygen consumption, if they are ingested the evening before the test. The activity proved to be comparable to that of Preparation I. Surprisingly, combined ingestion of both however produced a synergistic effect by decreasing heat production and oxygen consumption more than expected from the individual effects. Glucocorticoids did not produce such an effect, but rather increased heat production and oxygen consumption and prevented said effects of sulphonylurea derivatives and Preparation I, if ingested the evening before the test.

TABLE 4

| | | Mean | | |
|---|---|---|---|---|
| | | heart | Heat | Oxygen |
| | | rate | production | consumption |
| Test | Dosage | [per min.] | [kJ] | [liter] |
| A1-A5 | Control (averages | 126.7 | 1000 | 48 |
| | without dosage) | | | |
| A6 | 2.5 mg glibenclamide | 95.4 | 531 | 26 |
| A7 | 2.5 mg glibenclamide | 101.5 | 622 | 30 |
| A8 | 2.5 mg glibenclamide | 100.9 | 613 | 30 |
| A9 | 25 mg glibornuride | 100.0 | 600 | 29 |
| B10- | Control (averages | 137.5 | 2886 | 138 |
| B15 | without dosage) | | | |
| B16 | Prep. I | 124.7 | 2454 | 118 |
| B17 | Prep. I | 128.1 | 2568 | 123 |
| B18 | 10 mg chlorpropamide + | 122.2 | 2369 | 114 |
| | 5 mg gelatin | | | |
| B19 | 20 mg chlorpropamide + | 119.6 | 2281 | 109 |
| | 5 mg gelatin | | | |
| B20 | 100 μg glibenclamide + | 125.3 | 2474 | 119 |
| | 5 mg gelatin | | | |
| B21 | 200 μg glibenclamide + | 121.2 | 2335 | 113 |
| | 5 mg gelatin | | | |
| B22 | 1 mg glibornuride + | 121.6 | 2349 | 113 |
| | 5 mg gelatin | | | |
| B23 | 2 mg glibornuride + | 125.0 | 2464 | 118 |
| | 5 mg gelatin | | | |
| B24 | 10 mg chlorpropamide + | 85.6 | 1134 | 54 |
| | 5 mg gelatin + Prep. I | | | |
| B25 | 20 mg chlorpropamide + | 86.7 | 1171 | 56 |
| | 5 mg gelatin + Prep. I | | | |
| B26 | 100 μg glibenclamide + | 87.0 | 1181 | 57 |
| | 5 mg gelatin + Prep. I | | | |
| B27 | 200 μg glibenclamide + | 81.8 | 1006 | 48 |
| | 5 mg gelatin + Prep. I | | | |
| B28 | 1 mg glibornuride + | 98.0 | 1552 | 75 |
| | 5 mg gelatin + Prep. I | | | |
| B29 | 2 mg glibornuride + | 83.3 | 1056 | 51 |
| | 5 mg gelatin + Prep. I | | | |
| B30 | 20 μg dexamethasone + | 144.8 | 3132 | 150 |
| | 5 mg gelatin + Prep. I | | | |
| B31 | 200 μg prednisolone + | 145.5 | 3156 | 151 |
| | 5 mg gelatin + Prep. I | | | |
| B32 | 100 μg glibenclamide + | 135.0 | 2801 | 134 |
| | 5 mg gelatin + | | | |
| | 20 μg dexamethasone + | | | |
| | Prep. I | | | |
| B33 | 100 μg glibenclamide + | 133.6 | 2754 | 132 |
| | 5 mg gelatin + 200 μg | | | |
| | prednisolone + | | | |
| | Prep. I | | | |

c) In a further test series, the effect of Preparations I-IV was tested when applied in combination with sulphonylurea derivatives and/or glucocorticoids immediately before the physical exercise. In these tests, individual B was subjected to a workload of 125 W (60 r.p.m.) for 45 minutes; the drugs and Preparations were ingested 5 minutes before the test. The mean heart rate as well as the heat production and oxygen consumption induced by the workload are summarized in Table 5.

The results confirm that each of Preparations I-IV when taken alone significantly reduce heart rate, heat production and oxygen consumption. Sulphonylurea derivatives slightly increase heart rate, heat production and oxygen consumption (the diminished effect compared to Table 3 being explainable by the lower doses) and prevent the effect of Preparation I, except if taken in combination with a glucocorticoid. However, sulphonylurea derivatives do not prevent the effect of Preparations II-IV, but slightly improve their effect and especially that of Preparation IV. Glucocorticoids do not prevent the effect of Preparation I. In particular, it has however been found that simultaneous ingestion of glucocorticoids is suitable to significantly improve the effect of Preparations II-IV and that this improvement may be further increased by the additional intake of a glucocorticoid.

TABLE 5

| | | Mean | | |
|---|---|---|---|---|
| | | heart | Heat | Oxygen |
| | | rate | production | consumption |
| Test | Dosage | [per min.] | [kJ] | [liter] |
| 1-3 | Control (averages | 131.0 | 2666 | 128 |
| | without dosage) | | | |
| 4 | 20 mg chlorpropamide | 132.5 | 2717 | 130 |
| 5 | 1 mg glibornuride | 132.2 | 2706 | 130 |
| 6 | 100 μg glibenclamide | 132.6 | 2727 | 131 |
| 7 | Prep. I | 108.5 | 1907 | 92 |
| 8 | 20 mg chlorpropamide + | 130.2 | 2639 | 127 |
| | Prep. I | | | |
| 9 | 1 mg glibornuride + | 131.9 | 2697 | 129 |
| | Prep. I | | | |
| 10 | 100 μg glibenclamide + | 131.6 | 2658 | 130 |
| | Prep. I | | | |
| 11 | 20 μg dexamethasone + | 108.9 | 1920 | 92 |
| | Prep. I | | | |
| 12 | 200 μg prednisolone + | 109.4 | 1937 | 93 |
| | Prep. I | | | |
| 13 | 20 mg chlorpropamide + | 112.6 | 2045 | 98 |
| | 20 μg dexamethasone + | | | |
| | Prep. I | | | |
| 14 | Prep. II | 109.2 | 1930 | 93 |
| 15 | 20 mg chlorpropamide + | 105.1 | 1792 | 88 |
| | Prep. II | | | |
| 16 | 1 mg glibornuride + | 102.9 | 1718 | 82 |
| | Prep. II | | | |
| 17 | 100 μg glibenclamide + | 105.3 | 1799 | 88 |
| | Prep. II | | | |
| 18 | 20 μg dexamethasone + | 87.4 | 1195 | 57 |
| | Prep. II | | | |
| 19 | 200 μg prednisolone + | 84.2 | 1087 | 52 |
| | Prep. II | | | |
| 20 | 20 mg chlorpropamide + | 85.3 | 1124 | 54 |
| | 20 μg dexamethasone + | | | |
| | Prep. II | | | |
| 21 | Prep. III | 106.0 | 1822 | 87 |
| 22 | 100 μg glibenclamide + | 104.1 | 1758 | 84 |
| | Prep. III | | | |
| 23 | 20 μg dexamethsaone + | 95.0 | 1451 | 70 |
| | Prep. III | | | |
| 24 | 100 μg glibenclamide + | 88.1 | 1218 | 58 |
| | 20 μg dexamethasone + | | | |
| | Prep. III | | | |
| 25 | Prep. IV | 96.5 | 1513 | 73 |
| 26 | 100 μg glibenclamide + | 91.3 | 1326 | 64 |
| | Prep. IV | | | |
| 27 | 20 μg dexamethasone + | 81.6 | 999 | 48 |
| | Prep. IV | | | |
| 28 | 100 μg glibenclamide + | 79.7 | 935 | 45 |
| | 20 μg dexamethasone + | | | |
| | Prep. IV | | | |

1. A method of decreasing oxygen consumption in animal including human during physical work which comprises
administering simultaneously to said animal including human efficacious amounts (a) of a first component selected from the group consisting of D-glucose, D-maltose, ethanol, glucogenic amines, glucogenic amino acids, amino acids which can be metabolized via glyoxylate, and dipeptides and pharmaceutically acceptable salts of glucogenic amino acids and amino acids which can be metabolized via glyoxylate and (b) of a second component selected from the group consisting of thiamine, pharmaceutically acceptable thiamine salts and combinations of folic acid and cyanocobalamin, with the proviso that the second component is thiamine or a pharmaceutically acceptable thiamine salt if the first component is D-glucose, D-maltose, a glucogenic amine, a glucogenic amino acid which cannot be metabolized via glyoxylate, or a dipeptide or pharmaceutically acceptable salt of a glucogenic which cannot be metabolized via glyoxylate, and
thereby producing an effect of decreased oxygen consumption during the physical work.

2. The method as claimed in claim 1, wherein the first component is selected from the group consisting of D-glucose, D-maltose, glucogenic amines, glucogenic amino acids, amino acids which can be metabolized via glyoxylate, and dipeptides and pharmaceutically acceptable salts of glucogenic amino acids and amino acids which can be metabolized via glyoxylate, and the second component is selected from the group consisting of thiamine and pharmaceutically acceptable thiamine salts.

3. The method as claimed in claim 2, wherein the first component is administered in a dose of at least 1 g and the second component is administered in a dose of at least 10 mg.

4. The method as claimed in claim 2, wherein the first component is a fruit juice or fruit concentrate, comprising natural D-glucose in an efficacious amount.

5. The method as claimed in claim 2, wherein the first component is selected from the group consisting of L-aspartic acid, pharmaceutically acceptable L-aspartates, L-phenylalanine, L-tyrosine and L-tryptophan.

6. The method as claimed in claim 2, further comprising simultaneous administration of a vitamin selected from the group consisting of ascorbic acid, pharmaceutically acceptable ascorbates and cyanocobalamin.

7. The method as claimed in claim 1, wherein the first component is selected from the group consisting of amino acids which can be metabolized via glyoxylate, and dipeptides and pharmaceutically acceptable salts thereof, and the second component is a combination of folic acid and cyanocobalamin.

8. The method as claimed in claim 7, wherein the first component is selected from the group consisting of glycine, L-serine, L-glutamic acid, and dipeptides and pharmaceutically acceptable salts thereof.

9. The method as claimed in claim 7, wherein the first component is administered in a dose of at least 1 g, folic acid is administered in a dose of at least 0.2 mg and cyanocobalamin is administered in a dose of at least 1 μg.

10. The method as claimed in claim 1, wherein the first component is ethanol and the second component is a combination of folio acid and cyanocobalamin.

11. The method as claimed in claim 10, wherein ethanol is administered in a dose of at least 0.2 g, folic acid is administered in a dose of at least 0.2 mg and cyanocobalamin is administered in a dose of at least 1 μg.

12. The method as claimed in claim 1, wherein the first component is ethanol and the second component is selected from the group consisting of thiamine and pharmaceutically acceptable thiamine salts.

13. The method as claimed in claim 12, further comprising simultaneous administration of an efficacious amount of biotin.

14. The method as claimed in claim 13, wherein ethanol is administered in a dose of at least 0.2 g, the second component is administered in a dose of at least 5 mg and biotin is administered in a dose of at least 0.1 mg.

15. The method as claimed in claim 1, further comprising simultaneous administration of a medicament selected from the group consisting of insulin, sulphonylurea derivatives having antidiabetic activity, glucocorticoids, chlorprothixene and thioxanthene.

16. The method as claimed in claim 15, wherein the medicament is a glucocorticoid and the first component is selected from the group consisting of ethanol and dipeptides of amino acids which can be metabolized via glyoxylate, and wherein administration is effected shortly before or during the physical work.

17. The method as claimed in claim 15, wherein the medicament is an antidiabetic sulphonylurea derivative and the first component is D-glucose, and wherein administration is effected on the day before the physical work.

18. The method as claimed in claim 1, wherein at least one of the components is gelled utilizing a gelling agent.

19. The method as claimed in claim 1, comprising administering a composition comprising the first component and the second component.

20. The method as claimed in claim 19, wherein the composition is gelled utilizing a gelling agent.

21. The method as claimed in claim 20, wherein the first component is selected from the group consisting of D-glucose, D-maltose, ethanol and glucogenic amines, and the gelling agent is a gellable polymeric carbohydrate.

22. The method as claimed in claim 21, wherein the gellable polymeric carbohydrate is selected from the group consisting of agar-agar and pectin.

23. The method as claimed in claim 20, wherein the first component is selected from the group consisting of ethanol, glucogenic amines, glucogenic amino acids, amino acids which can be metabolized via glyoxylate, and dipeptides and pharmaceutically acceptable salts of glucogenic amino acids and amino acids which can be metabolized via glyoxylate, and the gelling agent is a gellable protein.

24. The method as claimed in claim 23, wherein the gellable protein is gelatin.

25. A method of decreasing oxygen consumption in animal including human during physical work which comprises
administering to said animal including human a composition comprising (a) an efficacious amount of a first component selected from the group consisting of D-glucose, D-maltose, ethanol, glucogenic amines, glucogenic amino acids, amino acids which can be metabolized via glyoxylate, and dipeptides and pharmaceutically acceptable salts of glucogenic amino acids and amino acids which can be metabolized via glyoxylate, (b) an efficacious amount of a second component selected from the group consisting of thiamine, pharmaceutically acceptable thiamine salts and combinations of folic acid and cyanocobalamin, with the proviso that the second component is thiamine or a pharmaceutically acceptable thiamine salt if the first component is D-glucose, D-maltose, a glucogenic amine, a glucogenic amino acid which cannot be metabolized via glyoxylate, or a dipeptide or pharmaceutically acceptable salt of a glucogenic amino acid which cannot be metabolized via glyoxylate, and (c) a gelling agent in gelled form, and
thereby producing an effect of decreased oxygen consumption during the physical work.

26. The method as claimed in claim 25, wherein the first component is selected from the group consisting of D-glucose, D-maltose and glucogenic amines, the second component is selected from the group consisting of thiamine and pharmaceutically acceptable thiamine salts, and the gelling agent is a gellable polymeric carbohydrate.

27. The method as claimed in claim 26, wherein the gellable polymeric carbohydrate is selected from the group consisting of agar-agar and pectin.

28. The method as claimed in claim 26, wherein the composition comprises at least 100 mg of the first component and at least 1 mg of the second component.

29. The method as claimed in claim 26, wherein the composition further comprises an efficacious amount of a vitamin selected from the group consisting of ascorbic acid, pharmaceutically acceptable ascorbates and cyanocobalamin.

30. The method as claimed in claim 25, wherein the first component is ethanol, the second component is selected from the group consisting of thiamine and pharmaceutically acceptable thiamine salts, and the gelling agent is selected from the group consisting of gellable polymeric carbohydrates and gellable proteins.

31. The method as claimed in claim 30, wherein the composition comprises at least 10 mg of ethanol and at least 0.5 mg of the second component.

32. The method as claimed in claim 30, wherein the composition further comprises an efficacious amount of biotin.

33. The method as claimed in claim 25, wherein the first component is ethanol, the second component is a combination of folic acid and cyanocobalamin, and the gelling agent is selected from the group consisting of gellable polymeric carbohydrates and gellable proteins.

34. The method as claimed in claim 33, wherein the composition comprises at least 10 mg of ethanol, at least 0.1 mg of folic acid, and at least 1 μg of cyanocobalamin.

35. The method as claimed in claim 25, wherein the first component is selected from the group consisting of glucogenic amino acids, amino acids which can be metabolized via glyoxylate, and dipeptides and pharmaceutically acceptable salts of glucogenic amino acids and amino acids which can be metabolized via glyoxylate, the second component is selected from the group consisting of thiamine and pharmaceutically acceptable thiamine salts, and the gelling agent is a gellable protein.

36. The method as claimed in claim 35, wherein the composition comprises at least 1 mg of the first component and at least 1 mg of the second component.

37. The method as claimed in claim 35, wherein the first component is selected from the group consisting of L-aspartic acid, pharmaceutically acceptable L-aspartates, L-phenylalanine, L-tyrosine and L-tryptophan.

38. The method as claimed in claim 25, wherein the first component is selected from the group consisting of amino acids which can be metabolized via glyoxylate and dipeptides and pharmaceutically acceptable salts of amino acids which can be metabolized via glyoxylate, the second component is a combination of folic acid and cyanocobalamin, and the gelling agent is a gellable protein.

39. The method as claimed in claim 38, wherein the composition comprises at least 1 mg of the first component, at least 0.1 mg of folic acid and at least 1 μg of cyanocobalamin.

40. The method as claimed in claim 38, wherein the first component is a dipeptide selected from the group consisting of H-Gly-Gly-OH, H-Ser-Ser-OH und H-Glu-Glu-OH, and wherein the composition comprises at least 1 mg of said dipeptide, at least 0.1 mg of folic acid and at least 1 μg of cyanocobalamin.

41. The method as claimed in claim 25, wherein the composition further comprises a efficacious amount of a medicament selected from the group consisting of insulin, sulphonylurea derivatives having antidiabetic activity, glucocorticoids, chlorprothixene and thioxanthene.

42. The method as claimed in claim 41, wherein the medicament is a glucocorticoid, the first component is selected from the group consisting of ethanol and dipeptides of amino acids which can be metabolized via glyoxylate and the gelling agent is selected from gellable polymeric carbohydrates and gellable proteins, and wherein administration of the composition is effected shortly before or during the physical work.

43. The method as claimed in claim 41, wherein the medicament is an antidiabetic sulphonylurea derivative, the first component is D-glucose, the second component is selected from the group consisting of thiamine and pharmaceutically acceptable thiamine salts and the gelling agent is a gellable polymeric carbohydrate, and wherein administration of the composition is effected on the day before the physical work.

44. The method as claimed in claim 43, wherein the gellable polymeric carbohydrate is selected from the group consisting of agar-agar and pectin.

45. The method as claimed in claim 43, wherein the wherein the composition comprises at least 100 mg of D-glucose and at least 1 mg of the second component.

46. The method as claimed in claim 43, wherein the composition further comprises an efficacious amount of a vitamin selected from the group consisting of ascorbic acid, pharmaceutically acceptable ascorbates and cyanocobalamin.

47. A composition for decreasing oxygen consumption during physical work, comprising (a) an efficacious amount of ethanol and (b) an efficacious amount of a combination of folic acid and cyanocobalamin or an efficacious amount of a combination of biotin and thiamine or a pharmaceutically acceptable thiamine salt.

48. The composition as claimed in claim 47, comprising ethanol in a dose unit of at least 0.2 g, folic acid in a dose unit of at least 0.2 mg and cyanocobalamin in a dose unit of at least 1 μg.

49. The composition as claimed in claim 47, comprising ethanol in a dose unit of at least 0.2 g, thiamine or a pharmaceutically acceptable thiamine salt in a dose unit of at least 5 mg and biotin in a dose unit of at least 0.1 mg.

50. A composition for decreasing oxygen consumption during physical work, comprising
(a) an efficacious amount of a first component selected from the group consisting of D-glucose, D-maltose, ethanol, glucogenic amines, glucogenic amino acids, amino acids which can be metabolized via glyoxylate, and dipeptides and pharmaceutically acceptable salts of glucogenic amino acids and amino acids which can be metabolized via glyoxylate,
(b) an efficacious amount of a second component selected from the group consisting of thiamine, pharmaceutically acceptable thiamine salts and combinations of folic acid and cyanocobalamin, with the proviso that the second component is thiamine or a pharmaceutically acceptable thiamine salt if the first component is D-glucose, D-maltose, a glucogenic amine, a glucogenic amino acid which cannot be metabolized via glyoxylate, or a dipeptide or pharmaceutically acceptable salt of such an amino acid, and
(c) a gelling agent in gelled form.

51. The composition as claimed in claim 50, wherein the first component is selected from the group consisting of D-glucose, D-maltose and glucogenic amines, the second component is selected from the group consisting of thiamine and pharmaceutically acceptable thiamine salts, and the gelling agent is a gellable polymeric carbohydrate.

52. The composition as claimed in claim 51, wherein the gellable polymeric carbohydrate is selected from the group consisting of agar-agar and pectin.

53. The composition as claimed in claim 51, wherein the composition comprises at least 100 mg of the first component and at least 1 mg of the second component.

54. The composition as claimed in claim 51, wherein the composition further comprises an efficacious amount of a vitamin selected from the group consisting of ascorbic acid, pharmaceutically acceptable ascorbates and cyanocobalamin.

55. The composition as claimed in claim 50, wherein the first component is ethanol, the second component is selected from the group consisting of thiamine and pharmaceutically acceptable thiamine salts, and the gelling agent is selected from the group consisting of gellable polymeric carbohydrates and gellable proteins.

56. The composition as claimed in claim 55, wherein the composition comprises at least 10 mg of ethanol and at least 0.5 mg of the second component.

57. The composition as claimed in claim 55, wherein the composition further comprises an efficacious amount of biotin.

58. The composition as claimed in claim 50, wherein the first component is ethanol, the second component is a combination of folic acid and cyanocobalamin, and the gelling agent is selected from the group consisting of gellable polymeric carbohydrates and gellable proteins.

59. The composition as claimed in claim 58, wherein the composition comprises at least 10 mg of ethanol, at least 0.1 mg of folic acid, and at least 1 μg of cyanocobalamin.

60. The composition as claimed in claim 50, wherein the first component is selected from the group consisting of glucogenic amino acids, amino acids which can be metabolized via glyoxylate, and dipeptides and pharmaceutically acceptable salts of glucogenic amino acids and amino acids which can be metabolized via glyoxylate, the second component is selected from the group consisting of thiamine and pharmaceutically acceptable thiamine salts, and the gelling agent is a gellable protein.

61. The composition as claimed in claim 60, further comprising an efficacious amount of a vitamin selected from the group consisting of ascorbic acid, pharmaceutically acceptable ascorbates, cyanocobalamin, pyridoxine and pharmaceutically acceptable pyridoxine salts.

62. The composition as claimed in claim 60, wherein the composition comprises at least 1 mg of the first component and at least 1 mg of the second component.

63. The composition as claimed in claim 50, wherein the first component is selected from the group consisting of amino acids which can be metabolized via glyoxylate and dipeptides and pharmaceutically acceptable salts of amino acids which can be metabolized via glyoxylate, the second component is a combination of folic acid and cyanocobalamin, and the gelling agent is a gellable protein.

64. The composition as claimed in claim 63, wherein the gellable protein is gelatin.

65. The composition as claimed in claim 63, wherein the composition comprises at least 1 mg of the first component, at least 0.1 g of folic acid and at least 1 μg of cyanocobalamin.

66. The composition as claimed in claim 65, wherein the first component is a dipeptide selected from the group consisting of H-Gly-Gly-OH, H-Ser-Ser-OH und H-Glu-Glu-OH.

67. The composition as claimed in claim 50, wherein the composition further comprises a efficacious amount of a medicament selected from the group consisting of insulin, sulphonylurea derivatives having antidiabetic activity, glucocorticoids, chlorprothixene and thioxanthene.

68. The composition as claimed in claim 67, wherein the medicament is a glucocorticoid, the first component is selected from the group consisting of ethanol and dipeptides of amino acids which can be metabolized via glyoxylate and the gelling agent is selected from gellable polymeric carbohydrates and gellable proteins.

69. The composition as claimed in claim 67, wherein the medicament is an antidiabetic sulphonylurea derivative, the first component is D-glucose, the second component is selected from the group consisting of thiamine and pharmaceutically acceptable thiamine salts and the gelling agent is a gellable polymeric carbohydrate.

70. The composition as claimed in claim 69, wherein the gellable polymeric carbohydrate is selected from the group consisting of agar-agar and pectin.

71. The composition as claimed in claim 69, wherein the wherein the composition comprises at least 100 mg of D-glucose and at least 1 mg of the second component.

72. The composition as claimed in claim 69, wherein the composition further comprises an efficacious amount of a vitamin selected from the group consisting of ascorbic acid, pharmaceutically acceptable ascorbates and cyanocobalamin.
